(19) United States
(12) Patent Application Publication
(10) Pub. No.: US 2008/0138444 A1
(43) Pub. Date: Jun. 12, 2008

(54) Method For Treating Bronchoconstriction and Pulmonary Vaso-Constriction

(75) Inventors: Stefan Weigand, Penzberg (DE); Reiner Frey, Wuppertal (DE); Johannes-Peter Stasch, Solingen (DE)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/664,882

(22) PCT Filed: Sep. 23, 2005
(86) PCT No.: PCT/EP05/10306

§ 371 Date: Dec. 21, 2007

(30) Foreign Application Priority Data
Oct. 5, 2004 (EP) 04023645.7

(51) Int. Cl. A61K 33/00; A61K 31/5377; A61K 31/506; A61K 31/135; A61P 11/08

(52) U.S. Cl. 424/718; 514/234.2; 514/256; 514/653

(57) ABSTRACT

This invention relates to the treatment and prevention of asthma or other forms of broncho-constriction or reversible pulmonary vasoconstriction in a mammal.

This invention relates to the treatment and prevention of asthma or other forms of broncho-constriction and reversible pulmonary vasoconstriction in a mammal.

Asthma is a chronic disease characterized by intermittent, reversible, widespread constriction of the airways of the lungs in response to any of a variety of stimuli which do not affect the normal lung. Estimates of the prevalence of this disease in the U.S. population range from three to six percent.

Drugs used to treat asthma fall generally into two categories: those which act mainly as inhibitors of inflammation, such as corticosteroids and cromolyn sodium, and those which act primarily as relaxants of the tracheobronchial smooth muscle, such as theophylline and its derivatives, beta-adrenergic agonists, and anticholinergics. Some of these bronchodilators may be administered orally, while others are generally given by intravenous or subcutaneous injection or by inhalation of the drug in an appropriate form, such as aerosolized powder (i.e., delivered in the form of a finely divided solid, suspended in a gas such as air), or aerosolized droplets (delivered in the form of a fine mist). Asthma patients typically self-administer bronchodilator drugs by means of a portable, metered-dose inhaler, employed as needed to quell or prevent intermittent asthma attacks.

Conceptually analogous to the narrowing of the airways of the lung which occurs in an asthma attack, vasoconstriction is a reversible narrowing of blood vessels attributable to contraction of the smooth muscle of the blood vessels. Such vasoconstriction can lead to abnormally high blood pressure (hypertension) in the affected portion of the circulatory system.

An elevation of the pulmonary arterial pressure (PAP) over normal levels is termed “pulmonary hypertension”.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hyper-tension is characterized by major structural changes in the pulmonary vasculature which result in a decreased cross-sectional area of the pulmonary blood vessels; this may be caused by, for example, chronic hypoxia, thromboembolism, or unknown causes (idiopathic or primary pulmonary hyper-tension).

Pulmonary hypertension has been implicated in several life-threatening clinical conditions, such as adult respiratory distress syndrome (“ARDS”) and persistent pulmonary hypertension of the newborn (“PPHN”). High resistance and low blood flow characterize the normal fetal pulmonary circulation. Pulmonary vascular resistance decreases dramatically during the normal transition from the fetal to neonatal circulation at birth. Mechanisms that explain the pulmonary vasodilatation at birth are incompletely understood but include alveolar ventilation, and the synthesis of vasoactive mediators such as nitric oxide (NO). NO plays an important role in the regulation of the developing pulmonary circulation by modulation of basal pulmonary vascular tone and reactivity in the late gestation fetus.

Pulmonary hypertension may also result in a potentially fatal heart condition known as “cor pulmonale”, or pulmonary heart disease.

Attempts have been made to treat pulmonary hypertension by administering drugs with known systemic vasodilatory effects, such as nitroprusside, hydralazine, and calcium channel blockers. Although these drugs may be successful in lowering the pulmonary blood pressure, they typically exert an indiscriminate effect, decreasing not only pulmonary but also systemic blood pressure. A large decrease in the systemic vascular resistance may result in dangerous pooling of the blood in the venous circulation, peripheral hypotension (shock), right ventricular ischemia, and consequent heart failure.

Physiological relaxation of blood vessels has been reported to result from the release of nitric oxide (NO) by endothelial cells lining the blood vessels. NO stimulates the enzyme guanylate cyclase within the vascular smooth muscle, with the resulting increase in cyclic GMP causing relaxation of this muscle, and thereby reversing vasoconstriction. NO is also believed to be produced by breakdown of organic nitrates such as nitroprusside and glyceryl trinitrate.

The present invention features methods for the prevention and treatment of asthma attacks or other forms of bronchoconstriction, of acute respiratory failure, or of reversible pulmonary vaso-constriction (i.e., acute pulmonary vasoconstriction or chronic pulmonary vasoconstriction which has a reversible component), in mammals (especially humans), which methods involve the steps of (1) identifying (by, for example, traditional diagnostic procedures) a mammal in need of such treatment or prevention; (2) causing the mammal to inhale a therapeutically-effective concentration of gaseous nitric oxide (or a therapeutically-effective amount of a nitric oxide-releasing compound); and (3) prior to, during or immediately after the NO-inhalation step, introducing into the mammal a therapeutically-effective amount of soluble guanylate cyclase (sGC) stimulator, preferably a compound selected from the group consisting of
2-[1-(2-fluorobenzyl)-1H-pyrazolo[3,4-b]pyridin-3-yl]-5-(4-morpholinyl)-4,6-pyrimidine-diamine (1), described also as example 16 in WO 00/06569, herein incorporated by reference,
2-[1-(2-fluorobenzyl)-1H-pyrazolo[3,4-b]pyridin-3-yl]-5-(4-pyridinyl)-4-pyrimidinamine (2), described also as example 1 in WO 02/42301, herein incorporated by reference,
methyl 4,6-diamino-2-[1-(2-fluorobenzyl)-1H-pyrazolo[3,4-b]pyridin-3-yl]-5-pyrimidinyl-(methyl)carbamate (3), described also as example 8 in WO 03/095451, herein incorporated by reference,
methyl-4,6-diamino-2-[1-(2-fluorobenzyl)-1H-pyrazolo[3,4-b]pyridin-3-yl]-5-pyrimidinyl-carbamate (4), described also as example 5 in WO 03/095451, herein incorporated by reference,
4-[((4-carboxybutyl)-{2-[(4-phenethylbenzyl)oxy]phenethyl}amino)methyl]benzoic acid (5), described also as example 8a in WO 01/19780, herein incorporated by reference.

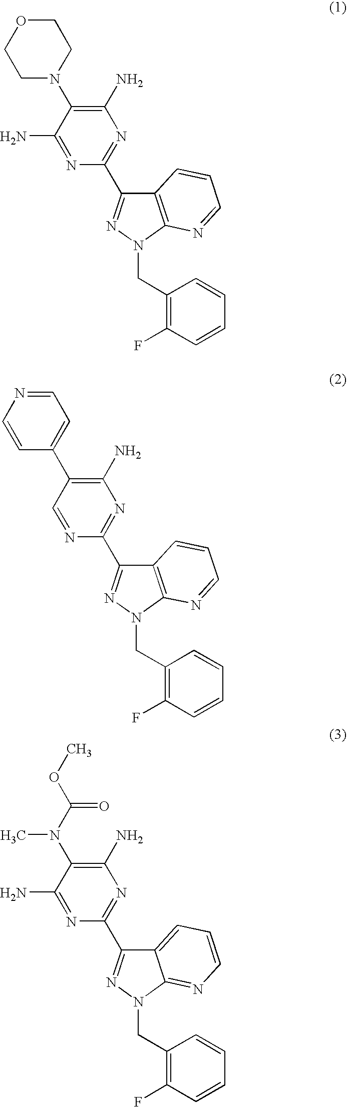

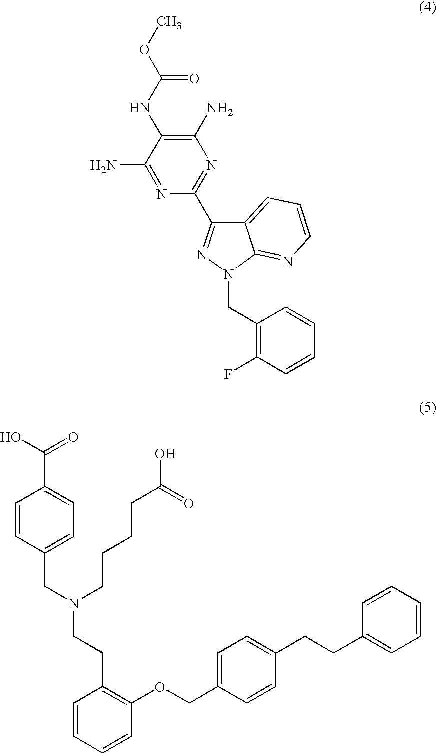

A “therapeutically effective” amount of a soluble guanylate cyclase (sGC) stimulator, preferably a compound selected from the group consisting of (1), (2), (3), (4), and (5), is herein defined as an amount which can increase the duration (i.e., half-time) of the therapeutic effect of gaseous NO or a NO-releasing compound by at least 100%.

Compounds (1), (2), (3) and (4) are soluble guanylate cyclase (sGC) stimulators which have been previously described for the treatment of stable angina pectoris or erectile dysfunction.

Treating or preventing bronchoconstriction or pulmonary hypertension with compound (1) or its inhalation as application/dosage form has not been described before.

Treating or preventing bronchoconstriction or pulmonary hypertension with compound (2) or its inhalation as application/dosage form has been described in DE 10310908.

Treating or preventing bronchoconstriction or pulmonary hypertension with compound (3) or (4) or their inhalation as application/dosage form has also been described in WO 03/095451.

The invention described herein provides a simple, safe, rapid, and efficacious treatment or preventative therapy for asthma attacks, for acute respiratory failure, and for both acute and certain forms of chronic pulmonary hypertension, without concomitantly lowering the systemic blood pressure of the patient. Pulmonary hypertension is a widespread clinical manifestation, afflicting diverse groups of patients.

Use of inhaled NO combined with soluble guanylate cyclase (sGC) stimulator, preferably a compound selected from the group consisting of (1), (2), (3), (4), and (5), treatment is currently envisioned for, but not limited to, preventing (if given prior to the onset of symptoms) or reversing acute pulmonary vasoconstriction, such as may result from pneumonia, traumatic injury, aspiration or inhalation injury, fat embolism in the lung, acidosis, inflammation of the lung, adult respiratory distress syndrome (ARDS), acute pulmonary edema, high altitude pulmonary edema (“mountain sickness”), asthma, post-cardiac surgery, acute pulmonary hypertension, persistent pulmonary hypertension of the newborn (PPHN), perinatal aspiration syndrome, hyaline membrane disease, acute pulmonary thromboembolism, acute pulmonary vasoconstriction in response to protamine reversal of heparin anticoagulation (“heparin-protamine reaction”), sepsis, status asthmaticus, or hypoxia (including that which may occur during one-lung anesthesia), as well as those cases of chronic pulmonary vasoconstriction which have a reversible component, such as may result from chronic pulmonary hypertension, bronchopulmonary dysplasia, chronic pulmonary thrombo-embolism, idiopathic or primary pulmonary hypertension, or chronic hypoxia.

Inhalation of gaseous nitric oxide represents a major advance in asthma therapy, since the gas has no particles or droplets to disperse and transport to the respiratory tract.

The soluble guanylate cyclase (sGC) stimulator, preferably a compound selected from the group consisting of (1), (2), (3), (4), and (5), may be introduced into the mammal by any suitable method, including via an oral, transmucosal, intravenous, intramuscular, subcutaneous, or intraperitoneal route.

The sGC stimulator, preferably a compound selected from the group consisting of (1), (2), (3), (4), and (5), may alternatively be inhaled by the mammal, in order to introduce it directly into the affected lung. In such a case, the sGC stimulator is advantageously formulated as a dry powder or as an aerosolized solution, having a particle or droplet size of less than 10 μm, for optimal deposition in the alveoli. Optionally, the soluble guanylate cyclase (sGC) stimulator, preferably a compound selected from the group consisting of (1), (2), (3), (4) and (5), can be inhaled in a gas containing gaseous nitric oxide.

The soluble guanylate cyclase (sGC) stimulator, preferably a compound selected from the group consisting of (1), (2), (3), (4) and (5), selected for use in the method of the invention may be administered as a powder (i.e., a finely divided solid, either provided pure or as a mixture with a biologically-compatible carrier powder, or with one or more additional therapeutic compounds) or as a liquid (i.e., dissolved or suspended in a biologically-compatible liquid carrier, optionally mixed with one or more additional therapeutic compounds), and can conveniently be inhaled in aerosolized form (preferably including particles or droplets having a diameter of less than 10 μm). Carrier liquids and powders that are suitable for inhalation are commonly used in traditional asthma inhalation therapeutics, and thus are well known to those who develop such therapeutics. The optimal dosage range can be determined by routine procedures by a pharmacologist of ordinary skill in the art.

In one embodiment of the invention, a portable inhaler equipped with a cartridge of compressed NO and an aerosol container of a soluble guanylate cyclase (sGC) stimulator, preferably a compound selected from the group consisting of (1), (2), (3), (4), and (5), in powder or liquid form could be used to administer inhalation therapy for asthma or for pulmonary vasoconstriction either in a hospital setting or in an emergency field situation. Such an inhaler can be carried, for example, by a person at risk of developing hypoxia, such as a mountain climber, or by ski patrol personnel who can administer the inhalation therapy on an emergency basis to skiers stricken with hypoxic pulmonary edema.

Determination of the preferred carrier (if any), propellant (which may include NO diluted in an inert gas such as N₂), design of the inhaler, and formulation of soluble guanylate cyclase (sGC) stimulator, preferably a compound selected from the group consisting of (1), (2), (3), (4), and (5), in its carrier are well within the abilities of those of ordinary skill in the art of devising routine asthma inhalation therapies. The portable inhaler could contain a canister of compressed NO, preferably in an inert carrier gas such as N₂, or any alternative means of providing NO gas. In addition, the inhaler could contain a soluble guanylate cyclase (sGC) stimulator, preferably a compound selected from the group consisting of (1), (2), (3), (4) and (5), either mixed in dry form with a propellant or held in a chamber separate from the propellant, or mixed with a liquid carrier capable of being nebulized to an appropriate droplet size, or in any other configuration known to those skilled in portable inhaler technology.

In another embodiment of the invention, a soluble guanylate cyclase (sGC) stimulator, preferably a compound selected from the group consisting of (1), (2), (3), (4) and (5), can be inhaled in the absence of gaseous nitric oxide for the treatment of the disorders described above, preferably for the treatment or prevention of bronchoconstriction or reversible pulmonary vasoconstriction in a mammal.

1. A method for treating or preventing bronchoconstriction or reversible pulmonary vaso-constriction in a mammal, which method comprises identifying a mammal in need of such treatment or prevention, causing said mammal to inhale a therapeutically-effective dose of gaseous nitric oxide, and prior to, during, or immediately after said inhalation step, introducing into said mammal a therapeutically-effective amount of a soluble guanylate cyclase (sGC) stimulator.

2. A method of claim 1, for the treating or preventing persistent pulmonary hypertension of the newborn.

3. The method of claim 1, wherein said sGC stimulator is a compound selected from the group consisting of (1), (2), (3) and (4).

4. The method of claim 1, wherein said sGC stimulator is a compound selected from the group consisting of (1), (2), (3), (4), and (5).

5. The method of claim 1, wherein said mammal is a human.

6. The method of claim 1, wherein said sGC stimulator is introduced into said mammal by an oral, intravenous, intramuscular, subcutaneous, or intraperitoneal route.

7. The method of claim 1, wherein said sGC stimulator is introduced into said mammal by causing said mammal to inhale an aerosol or dry powder comprising said sGC stimulator.

8. The method of claim 5, wherein said sGC stimulator is inhaled in a gas mixture comprising gaseous nitric oxide.

9. The method of claim 1, wherein said bronchoconstriction is associated with asthma.

10. A method for treating or preventing bronchoconstriction or reversible pulmonary vaso-constriction in a mammal, which method comprises identifying a mammal in need of such treatment or prevention, causing said mammal to inhale a therapeutically-effective dose of a soluble guanylate cyclase (sGC) stimulator.

11. The method of claim 10, wherein said sGC stimulator is a compound selected from the group consisting of (1), (2), (3), (4), and (5).

12. The method of claim 10, wherein said mammal is a human.

13. The method of claim 10, wherein said sGC stimulator is introduced into said mammal by causing said mammal to inhale an aerosol or dry powder comprising said sGC stimulator.

14. The method of claims 1 and 10, wherein said bronchoconstriction is associated with asthma.
